(19) United States
(12) Patent Application Publication
(10) Pub. No.: US 2014/0276422 A1
(43) Pub. Date: Sep. 18, 2014

(54) BULK FLUID SOURCE INJECTOR SYSTEMS

(71) Applicant: MEDRAD, INC., Indianola, PA (US)

(72) Inventors: David M. REILLY, Pittsburgh, PA (US); Edward J. RHINEHART, Monroeville, PA (US)

(73) Assignee: MEDRAD, INC., Indianola, PA (US)

(21) Appl. No.: 13/804,500

(22) Filed: Mar. 14, 2013

(51) Int. Cl. A61J 1/20; A61M 5/142; A61M 5/14

(52) U.S. Cl. CPC A61J 1/20; A61M 5/14; A61M 5/142
USPC 604/151; 604/411; 604/403; 604/93.01

(57) ABSTRACT

An injector system including a docking station and a cassette and various transfer devices for transferring fluid from a bulk fluid container to a reservoir in the cassette are described herein along with methods for using such devices and systems to inject fluid from the bulk fluid container into a patient.

### BACKGROUND

Sources that provide contrast are therefore provided by manufacturers in numerous concentrations in sterilized containers (such as glass bottles or plastic packages) ranging incrementally in size from 20 ml to 200 ml. These containers are generally designed for a single use in which once a container is opened for a patient, then it is used for that patient only. The contrast is generally aspirated from such containers via a syringe pump used to inject the contrast, and any contrast remaining in the container is discarded to prevent infection with potentially contaminated contrast. The medical staff is faced with the task of choosing an appropriately sized contrast container to assure an adequate injection while minimizing discarded contrast. Time consuming procedures are required to reload the syringe if more contrast is required than originally calculated. On the other hand, expensive waste results if only a portion of a filled syringe is injected. The inventory of contrast containers required under the current system increases costs and regulatory burdens throughout the contrast media supplier-consumer chain.

### SUMMARY OF THE INVENTION

Embodiments described herein are generally directed to an injector system including a docking station that at least includes a means for holding one or more bulk fluid containers and a cassette configured to be removeably connected to the docking station. The cassette of some embodiments may include a cassette body, one or more reservoirs dispose within the cassette body, at least one inlet port operably connected to each of the one or more reservoirs, at least one fluid passage operably connected to each of the one or more reservoirs, and an outlet port at a convergence of at least one fluid passage. In certain embodiments, the injector system may include a transfer device configured to operably connect at least one bulk fluid container held by the docking station to the at least one inlet port of the cassette.

Other embodiments are directed to cassettes for transporting fluid from a source to a patient that includes a cassette body, one or more reservoirs dispose within the cassette body, at least one inlet port operably connected to each of the one or more reservoirs, at least one fluid passage operably connected to each of the one or more reservoirs, and an outlet port at a convergence of at least one fluid passage.

Still other embodiments are directed to transfer devices including a spike, a port connector, a transfer tube operably connected to the spike and the port connector, and a guard enclosing the spike, port connector and transfer tube.

### BRIEF DESCRIPTION OF THE DRAWINGS

In the following detailed description, reference is made to the accompanying drawings, which form a part hereof. In the drawings, similar symbols typically identify similar components unless context dictates otherwise. The illustrative embodiments described in the detailed description, drawings, and claims are not meant to be limiting. Other embodiments may be utilized and other changes may be made, without departing from the spirit or scope of the subject matter presented herein. It will be readily understood that the aspects of the present disclosure, as generally described herein and illustrated in the figures, can be arranged, substituted, combined, separated, and designed in a wide variety of different configurations, all of which are explicitly contemplated herein.

FIG. 1 is a drawing showing a fluid delivery system including a docking station and a cassette.

FIG. 2 is a drawing showing the external features of a cassette.

FIG. 3 is a drawing showing a cut-a-way of the cassette.

FIG. 4 is a drawing showing a pumping unit incorporated into the cassette and a drive motor integrated into the docking station contacting the pumping unit.

FIG. 5 is a drawing showing alignment of the inlet port of the cassette, the bulk fluid container, and a transfer device.

FIG. 6 A-D are drawings showing transfer devices having a retractable inlet port connectors.

FIG. 7 A-D are drawings showing transfer devices having non-retractable inlet port connectors.

### DETAILED DESCRIPTION OF THE INVENTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this document is to be construed as an admission that the embodiments described in this document are not entitled to antedate such disclosure by virtue of prior invention.

This disclosure is not limited to the particular systems, devices and methods described, as these may vary. The terminology used in the description is for the purpose of describing the particular versions or embodiments only, and is not intended to limit the scope.

The word “proximal” refers to a direction relatively closer to a clinician using the device described herein, and the word “distal” refers to a direction relatively further from the clinician. For example, the end of a catheter placed within the body of a patient is considered a distal end of the catheter, while the catheter end remaining outside the body is a proximal end of the catheter.

Embodiments described herein are directed to systems and devices for transferring fluids from a bulk fluid source to a patient. As illustrated in FIG. 1, such systems 1 may at least include a docking station 10, a cassette 12 removably incorporated into the docking station 10, and a fluid transfer connector 14. The docking station 10 typically includes one or more bulk source containers 102 that are fluidly attached to the cassette 12 by a fluid transfer connector. This attachment allows fluid from the bulk fluid containers to be transferred to reservoirs in the cassette. The cassette includes various fluid transfer tubes and passage ways that lead to a cassette internal pump that transfers fluids from the cassette to a delivery port 104 on the docking station that can be operably connected to tubing to carry the fluid to a patient. When reservoirs in the cassette are emptied or nearly emptied the cassette can be removed and replaced.

The systems and devices of such embodiments allow for safe transfer of fluids from a bulk source container through the system for final delivery to the patient with reduced contamination from either the operator of the device or patient to patient contamination from multiple use patient delivery tubing. In addition, the systems and devices described above can be used as an injection system for continuous delivery of fluids to multiple patients. Operation of the systems and devices may be automatic to minimize user contact with the bulk source container further reducing the likelihood of contamination.

The docking station 10 of various embodiments may be designed to receive a cassette 12 and may include one or more holders for bulk fluid containers 106, hooks for holding bagged fluids 108 or combinations thereof. The docking station system of embodiments may further include, for example, sensors, detectors, actuators, pumps, heating systems, control systems, user interfaces, and the like that facilitate connection with the cassette, monitor fluid levels in the cassette and bulk fluid containers, and the like. These various components will be described in relation to the cassette 12 below.

The cassette 12 of various embodiments may include any number of components. FIG. 2 shows one example of a cassette 22 of some embodiments and illustrates the external features of the cassette 22. This cassette 22 includes a cassette body 202 shaped to be received by the docking station, one or more inlet port 204 positioned to allow the internal reservoirs of the cassette to be filled, at least one vent 206 associated with each reservoir and positioned to allow air or other gases in the reservoir to be removed as the reservoir is filled, and at least one exit port 208 positioned to communicate with the delivery port 104 of the docking station 10. In some embodiments, the cassette 22 may further include on or more connector including a guard and pierceable septum 210 that is capable of receiving a needle or other spiked connector. The external surfaces of the cassette 22 may further include various grooves or other mechanical alignment features that allow for easy connection with the docking station and handles or grooves for allowing ease of uses by a user.

FIG. 3 shows a cross section of a cassette of some embodiments. In such embodiments, at least one reservoir may be disposed within the cassette body. In some embodiments, the cassette body includes a least a first reservoir 312a, a second reservoir 312b, and a third reservoir 312c for holding a medicament or other medical fluid. Each reservoir may include a separate inlet port 304/310 and at least one air vent 306. The cassette may further include various fluid passages 314 disposed within the cassette body 32 in fluid communication with each reservoir 312a, 312b, 312c. In some embodiments, the fluid passages 314 may lead to an exit port, and in certain embodiments, each fluid passage my lead to a separate exit port that can deliver fluid to the docking station where it may be combined and delivered to a patient. In other embodiments, the fluid passages may carry fluids from the reservoirs to a pumping unit (not shown) disposed within the cassette. Such an internal pumping unit may be capable of drawing fluids from each reservoir individually and pumping the fluid through a common exit port and to the delivery port 104 on the docking station 10. Thus, in particular embodiments as illustrated in FIG. 3, the pumping unit may include at least three pumping units or one pumping unit for each reservoir.

In particular embodiments, the cassette 32 may further include a waste reservoir 316 disposed within the cassette. The waste reservoir 316 may be operably connected to at least one fluid passage 314, and in certain embodiments, at least one outlet of the pumping unit may fluidly connect to the waste reservoir 314. In some embodiments, an exit port of emptying fluid in the waste reservoir 316 may be operably connected to the waste reservoir. In other embodiments, the waste reservoir may hold waste fluid throughout operation and waste fluid may be discarded with the cassette. Thus, no exit port is necessary in such embodiments. In certain embodiments, the waste reservoir 316 may include at least one air vent 318 that may allow for the release of air as the waste reservoir 316 is filled with waste fluid.

In some embodiments, the cassette 32 may include one or more valves arranged to control transfer of fluid through the cassette 32 and into the docking station and delivery port. For example, in particular embodiments, one or more valves such as, for example, a rotary valve, pinch valve, spool valve, trumpet valve, or combinations thereof may be positioned along a fluid path between the reservoirs 312a, 312b, 312c and the exit port. Such valves may regulate the flow of fluid from each reservoirs 312a, 312b, 312c to the exit port allowing transfer of fluids from each bulk source container to be individually controlled. In other embodiments, one or more valves may be positioned at the exit port. For example, in particular embodiments, the exit port may include a three way valve that allows fluid from reservoirs 312a, 312b, 312c to enter the delivery port simultaneously or individually. In other embodiments, such a three way valve may deliver fluids from two reservoirs 312a, 312b, 312c and the exit port into a mixing chamber associated with the delivery port that allows for mixing of the fluids before the fluids are pumped through the exit port. In further embodiments, the various valves described above can be combined to control fluid flow from three, four, five, six, or more reservoirs.

The air vents 306 may be any type of air vent known in the art that can allow for exhaust of air from the reservoir or channel including, for example, a float valve. In some embodiments, the air vent 306 may also include filter that is disposed on the external opening of the air vent 306 to minimize any air born particulates from entering the reservoirs 312 through the air vents 306. In particular embodiments, the air vents 306 may allow flow of air out of the reservoir, without restricting the filling rate of the fluid from the bulk fluid container, which may be about 2 ml/sec or more. When injecting the air vents 306 must allow air into the cassette as fast as the injection, for example, at about 10 ml/sec or more, without causing a pressure drop in the reservoir which impacts the injection rate.

In particular embodiments, one or both of the first reservoir 312a, and second reservoir 312b may hold contrast fluid for injection into a patient, and the third reservoir 312c may hold saline. In operation, the pumping unit may allow for these fluids to be combined before delivery to the patient allowing contrast fluids to be mixed and/or diluted as necessary. In other embodiments, saline from the third reservoir 312c may be used to prime the fluid path to the patient before administration of contrast agent, and clear the fluid path after delivery to ensure that the patient receives a safe and complete dose of contrast agent.

Referring again to FIG. 1, in some embodiments, the cassette 12 may slide into an opening on a side of the docking station 10. The cassette 12 and the docking station 10 may be designed such that the cassette 12 can only fit into the docking station 10 in one way. For example, the cassette 12 may have a trapezoidal shape on one or more sides, and the shape of the cassette 12 may dictate that the cassette 12 is properly aligned in the docking station 10. In other embodiments, grooves or other mechanical features may be milled into the cassette 10, and these grooves may align with protrusions or other mechanical features included in the docking station. These interlocking grooves and protrusions may allow for proper alignment of the cassette in the docking station. In still other embodiments, the docking station 10 may include an actuator mechanism that receives the cassette 12 and aligns it within the docking station 10.

Once introduced into the docking station 10, the cassette 12 may be retained in position by friction or the docking station may include, for example, a lever or clamping mechanism to hold the cassette 12 in position. In other embodiments, the lever may be connected to cams, which force pins to contact the bottom of the cassette, and in certain embodiments, the pins may contact a flange of the pump forcing it to the top of the slot thereby preventing it from moving in any direction. Once the cassette system is secured to the docking system then fluid can be delivered through the cassette system to the reservoir.

In particular embodiments, the docking station 10 may include drive motor designed to operably connect to the pumping unit of the cassette. FIG. 4 shows an example of a cassette 42 including one or more pistons (3 pistons) 441 contained within adjacent bores 440 having at least one inlet from the fluid passages in the cassette and at least one outlet to a fluid passage leading to the exit port. A drive motor 442 assembly including a rotor 444 and at least one cam 446 per piston is illustrated below the cartridge and is encased in the docking station. For ease of understanding, the housing and other portions of the docking station have been omitted from the drawing. The cassette 42 is illustrated as being positioned in the docking station, when properly positioned, each cam 446 is aligned with the pistons 441 in the cartridge 42 and contacts a bottom surface of the piston 441. As the rotor 444 is rotated by the drive motor 442, the cams 446 also rotate and their oblong shape causes the pistons 441 to move up and down in the bores 449 causing fluid to be pumped from the reservoirs 412a, 412b, 412c in the cassette 42 to the exit port of the cassette and into the docking station to the patient delivery port.

The cam 446 may drive the pistons 441 to provide a specific flow profile. For example, in embodiments as illustrated in FIG. 4 in which the pumping unit includes three pistons, the flow output may be constant with little pressure variation and with little pressure drop between the reservoir and the pump so that it can draw without a pressure drop near atmospheric pressure. This allows for smooth fluid flow matching the smoothness of a syringe type injector. While embodiments are not limited to particular flow rates and pressures, in some embodiments, the drive motor and pumping unit may allow for pumping at a flow rate of about 5 ml/sec to about 25 ml/sec, about 7 ml/sec to about 15 ml/sec, about 8 ml/sec to about 10 ml/sec or about 10 ml/sec. The pressure produced by the drive motor and pumping station may be from about 200 psi to about 500 psi or 250 psi to about 400 psi and, in certain embodiments, about 300 psi.

In some embodiments, the docking station 10 may include one or more fluid detectors may also be provided in the docking system positioned to sense or measure the fluid being transferred into or out of the reservoirs of the cassette 12. Such fluid detectors can be positioned at any location along the fluid path. For example, in some embodiments, a fluid detector may be positioned at an outlet of each bulk fluid source 102 to measure a quantity of fluid dispensed from the bulk fluid source 102. In other embodiments, a fluid detector may be positioned at reservoirs to measure a quantity of fluid in the reservoirs. For example, a prism may be provided in the side of the cassette and sensors in alignment with the cassette when it is positioned and locked docking station may detect and quantify fluid moving through the cassette. In further embodiments, fluid detectors may be positioned at various locations along the fluid path. The fluid detectors of such embodiments may be any detector capable of determining or measuring the presence or amount of fluid moving through the detector.

The docking station may additionally include a control system that can include, for example, a graphic user interface (GUI) 110 or monitor that can be used as both an output and an input (i.e., a touch screen) or that may be used solely as an output device. The docking station may further include one or more input means such as, for example, a keyboard, remote control receiver and remote control, Internet, wifi, Bluetooth connection means for receiving input from an off site processor or central computing system, and the like and combinations thereof. In certain embodiments, the control system may be capable of stopping fluid flow under conditions where fluid should not be flowing, for example, if the cassette system is not installed properly in the docking system or when the fluid delivery system is not operating, thereby preventing any overflow or excessive flow of the fluid through the system.

In some embodiments, sensors may be incorporated into the cassette dock 106 may include one or more sensors to ensure proper alignment of the cassette 12 in the docking station 10. For example, in some embodiments, the cassette 12 may cooperate with a mechanical or optical switch on the cassette dock when properly aligned, and the control system may halt operation of the docking system unless the switch is activated. In other embodiments, the cassette may be encoded, and sensors on the docking station 10 in communication with the control system that can relay encoded information to the control system when the cassette is properly aligned. Such sensors verify that proper installation of the cassette 12 is maintained that the cassette 12 is secured during operation of the docking station. Various encoding means are known and can be used in such embodiments including, for example, radio frequency identification (RFID) encoding, optical encoding, magnetic encoding, mechanical encoding, and combinations thereof, and sensors specific to such encoding means may be incorporated into the docking station 10. In still other embodiments, the docking station 10 may include clamp positioning sensors such as a switch or rotary encoder to ensure proper position of a clamp or other mechanical locking apparatus used to maintain the position of the cassette 12 in the docking station 10. In further embodiments, the docking station may include level sensors and other sensors that ensure that the docking station and/or cassette properly aligned and level before operation.

In some embodiments, a fluid sensor may be located in the wall of the cassette that can determine the level of fluid in one or more reservoir. The sensor may become electronically coupled with a receiver located in the docking station when the cassette is properly positioned in the docking station. In some embodiments, each reservoir in the cassette may include sensor and a receiver for each sensor may be positioned at an appropriate position on the docking station, and in other embodiments, a single contact may be provided that allows information from more than one sensor to be transmitted from the cassette to the docking station. Any type of sensor can be used in various embodiments such as, for example, a reflective optical sensor.

In particular embodiments, the docking station may include a heating system positioned to heat portions of the cassette near reservoirs to maintain a predetermined temperature of the fluids in the reservoirs. For example, in some embodiments, the heating system may be embedded into the portion of the docking structure where the cassette sits when in place to put the cassette reservoirs in close proximity of the heater for efficient heating. The heaters may be any type of heater known in the art such as, for example, microwave or immersion heaters, and heating may automatically occur when the system is in operation of when fluid is in the reservoirs. Thus, the heaters may be activated by the control system, which can receive information regarding fluid levels from fluid level sensors.

In some embodiments, the docking station 10 may include a waste container in fluid communication with the filling port 104 or the receptacle 122, and in certain embodiments, the waste container may be integral to the cassette 12. Communication of the waste container with the filling port 104 or receptacle 122 can be facilitated by a valve, or in some embodiments, waste fluids may be contained within the receptacle 122 and not delivered to a patient. A user may then discard the waste fluid by removing the receptacle from the cassette 12 or by discarding the cassette 12.

The bulk fluid storage container may contact the cassette by any means. For example, in some embodiments, standard IV tubing may be used to connect a bulk fluid container to a reservoir. In such embodiments, a needle may be disposed at the proximal end of a transfer tube, and this needle may be received by a septum on the cassette (see medical fluid reservoir FIGS. 2 and 3).

In other embodiments, a transfer device may be placed between the bulk fluid container and the cassette. As illustrated in FIG. 5, in such embodiments, in inlet port 504 of the cassette 52 may substantially align with an outlet 532 of the bulk fluid container 53 when the cassette 52 is properly positioned in the docking station. A transfer device 55 may be fluidly connected to the outlet 532 of the bulk fluid container 53 and fluidly connected to the inlet port 504 of the cassette 52 allowing for fluid from the bulk fluid container 53 to be transferred to a reservoir in the cassette 52. In some embodiments, the transfer device 55 may have a generally cylindrical shape and can be composed of a rigid or semi-rigid material. The transfer device 55 may include various components for facilitating transfer of fluids such as, for example, a guard 553, a spike 554 for piercing a septum associated with the outlet 532 of the bulk fluid container 53, a connector 556 for connecting with the inlet port 503 of the cassette 52, transfer tubing 558 disposed within the guard 553, an transfer device air vent 557, and the like an various combinations thereof.

FIG. 6 shows a more specific example of a transfer device 65. The transfer device 65 of FIG. 6 includes a guard 653, spike 654, an inlet port connector 656, and transfer tubing 658. The inlet port connector 656, in such embodiments, includes the pierceable septum 657 attached to a retractable connector 656 that is slidably connected to the guard 653 by a spring 655. The guard 653 may be sized and shaped to removeably connect with a portion of the inlet port 604 of the cassette 60. In operation as illustrated in FIG. 6B, the proximal portion of the guard 653 may be received by distal extension 605 of the inlet port 604 of the cassette 60 allowing the transfer device septum 657 to contact an inlet port septum 603. The retractable connector 656 may retract when this connection is made allowing the transfer tubing 658 to pierce both the transfer device septum 657 and the inlet port septum 603 permitting fluid to be transferred from the bulk fluid container to the reservoir in the cassette 60 through the transfer device 65.

FIG. 6C and FIG. 6D show a similar arrangement of components. However, in this embodiment, the inlet port septum 603 is connected to the cassette using a retractable extension 607 and spring 609 housing a cassette transfer tube 601. The transfer device septum 657 may be fixedly attached to the transfer tubing 658 of the transfer device 65. The transfer device septum 657 and the inlet port septum 603 may be made from any material, and in certain embodiments, these septum may be composited of an elastomeric material, such as, but not limited to, silicone rubber, EPDM, Buna-N, VINTON, and the like, that can be pierced and sealable connect the inlet port connector 756 and the inlet port 704. In operation as illustrated in FIG. 6C, the proximal portion of the guard 653 may be received by distal extension 605 of the inlet port 604 of the cassette 60 allowing the transfer device septum 657 to contact an inlet port septum 603. The retractable inlet connector 607 may retract when this connection is made allowing the inlet transfer tube 601 to pierce both the transfer device septum 657 and the inlet port septum 603 permitting fluid to be transferred from the bulk fluid container to the reservoir in the cassette 60 through the transfer device 65.

FIG. 7 shows another embodiment of the transfer device 75 in which retractable part of the inlet port 704 have been eliminated. Such transfer devices 75 include a guard 753, spike 754, an inlet port connector 756, and transfer tubing 758. In operation, the proximal portion of the guard 753 may be received by distal extension 705 of the inlet port 704 of the cassette 70 allowing the transfer device septum 757 to contact an inlet port septum 703. As illustrated in FIG. 7A, the distal end of the inlet port connector 756 may be shaped to directly connect to the proximal portion of the inlet port 704, and in certain embodiments, inlet port extensions 759 may facilitate proper alignment of inlet port connector 756 and the inlet port 704. The inlet port connector 756 and the inlet port 704 can electrometrically connect these components creating a “face seal.” Connection of the inlet port connector 756 and the inlet port 704 may permit fluid to be transferred from the bulk fluid container to the reservoir in the cassette 70 through the transfer device 75.

FIGS. 7C and D show another example of a transfer device 75 that is configured to create a direct inlet port connector 756 and the inlet port 704. Here, a retractable inlet port connector 756 may include an internal spring 755 and a two part transfer tubing system in which a first part of the transfer tube 758a is fixedly attached to a spike 754 and a second part of the transfer tube 758b is contained within the retractable inlet port connector 756. In such embodiments, when the retractable inlet port 756 is extended, i.e., when the transfer device 75 is not in contact with the cassette 70, the outlet of the first part of the transfer tube 758a is blocked by the retractable inlet port 756 stopping fluid flow, as illustrated in FIG. 7D. Upon connection, the inlet port connector 756 and the inlet port 704 create a operable connection. The spring 759 may be compressed allowing the outlet of the first part of the transfer tube 758a to align with the opening of the second part of the transfer tube 758b allowing fluid to flow through the transfer device 75 and into the reservoir of the cassette 70 through the connection between the inlet port connector 756 and the inlet port 704.

As illustrated in FIGS. 6C, 6D, 7A, 7B, 7C, and 7D, various embodiments of the transfer device 65, 75 may include an air vent 650, 750, and the air vent 650, 750 may be configured to allow air to enter the bulk fluid container. As such, a portion of the spike 654, 754 may be operably connected to the air vent 650, 750 and may provide a secondary passage into the bulk fluid container. Thus, in operation, as fluid exits the bulk fluid container through the primary passage of the spike 654, 754, through the transfer tubing 658, 758 and into the reservoir in the cassette 60, 70, air may be allowed to enter the bulk fluid container from the air vent 650, 750 through the secondary passage. In other embodiments, a vent tube may be separately provided in the transfer device 65, 75 that can be supported by at least a portion the spike 754. The vent tube can terminate above or below the entrance to the fluid drain tube. Such an arrangement allows for continuous flow of fluid by reducing or eliminating a negative pressure in the bulk fluid container that could stop fluid flow. In certain embodiments, the air vent may include a check valve, vent filter, or combinations thereof.

In some embodiments as illustrated in FIGS. 6C, 6D, 7A, and 7B, the transfer device 65, 75 may include a fluid drain 651, 751 disposed in the guard 653, 753 between the spike 654, 754 and inlet port connector 656, 756. Fluid drain 651, 751 may include a flanged tube that allows a volume of fluid to collect in the transfer device 65, 75 to avoid overflow.

In still other embodiments, an overflow check value may be provided in the transfer device 65, 75. For example, a ball valve may open when liquid rises the ball floats and close off the a hole in the valve creating an electrometric seal and preventing fluid leaking out as fluid is withdrawn from the reservoir the ball drops and allows makeup air to enter the reservoir.

Although the present invention has been described in detail in connection with the above embodiments and/or examples, it should be understood that such detail is illustrative and not restrictive, and that those skilled in the art can make variations without departing from the invention. The scope of the invention is indicated by the following claims rather than by the foregoing description. All changes and variations that come within the meaning and range of equivalency of the claims are to be embraced within their scope.

What is claimed is:

1. A transfer device comprising:
a spike;
a port connector;
a transfer tube operably connected to the spike and the port connector; and
a guard enclosing the spike, port connector and transfer tube.

2. The transfer device of claim 1, further comprising an air vent and a vent tube or secondary passage aligned with the spike.

3. The transfer device of claim 2, further comprising a check valve, air filter, or combination thereof operably connected to the air vent.

4. The transfer device of claim 1, wherein the port connector is retractable an operably connected to a spring.

5. The transfer device of claim 4, wherein the retractable port connector block flow of fluid through the transfer tube when in extended position.

6. The transfer device of claim 4, further comprising a first transfer tube operably connected to the spike and a second transfer tube enclosed within the port connector.

7. The transfer device of claim 1, further comprising a septum disposed over a distal portion of the transfer tube.

8. A cassette for transporting fluid from a source to a patient comprising:
a cassette body;
one or more reservoirs dispose within the cassette body;
at least one inlet port operably connected to each of the one or more reservoirs;
at least one fluid passage operably connected to each of the one or more reservoirs; and
an outlet port at a convergence of at least one fluid passage.

9. The cassette of claim 8, further comprising at least one air vent operably connected to each of the one or more reservoirs.

10. The cassette of claim 8, further including a waste reservoir.

11. The cassette of claim 8, further comprising a fluid detector operably connected to at least one of the one or more reservoirs.

12. The cassette of claim 8, further comprising a pumping unit disposed within the cassette body.

13. The cassette of claim 8, further comprising as air detector operably connected to the outlet port.

14. An injector system comprising:
a docking station, the docking station at least comprising a means for holding one or more bulk fluid containers; and
a cassette configured to be removeably connected to the docking station, the cassette comprising:
a cassette body;
one or more reservoirs dispose within the cassette body;
at least one inlet port operably connected to each of the one or more reservoirs;
at least one fluid passage operably connected to each of the one or more reservoirs; and
an outlet port at a convergence of at least one fluid passage.

15. The injector system of claim 13, further comprising a transfer device configured to operably connect at least one bulk fluid container held by the docking station to the at least one inlet port of the cassette.

16. The injector system of claim 14, wherein the transfer device comprises:
a spike;
a port connector;
a transfer tube operably connected to the spike and the port connector; and
a guard enclosing the spike, port connector and transfer tube.

17. The injector system of claim 13, wherein the docking station further comprises graphical user interface or monitor and a control system.

18. The injector system of claim 16, wherein the cassette further comprises a pumping unit and the docking station further comprises a drive motor operably connected to the control system, the drive system being configured to be operably connected to the pumping unit.

19. The injector system of claim 16, wherein the docking station further comprises one or more sensors operably connected to the control system.

20. The injection system of 13, further comprising a heater.
